SEQUENCE LISTING (32 sequences)

SEQ ID NO: 1 - DNA, 20 bp, artificial sequence
  primer
cacaggatacacagagaatg

SEQ ID NO: 2 - DNA, 20 bp, artificial sequence
  primer
cttgatggcgatgaatttag

SEQ ID NO: 3 - DNA, 24 bp, artificial sequence
  probe
attggaagtcatgcagcacatggc

SEQ ID NO: 4 - DNA, 21 bp, artificial sequence
  primer
ggacattgataacgaagattg

SEQ ID NO: 5 - DNA, 18 bp, artificial sequence
  primer
gctgcctgagatactgat

SEQ ID NO: 6 - DNA, 23 bp, artificial sequence
  probe
agaaccctcagctctgcagtgac

SEQ ID NO: 7 - DNA, 15 bp, artificial sequence
  primer
ggcactcttgccttc

SEQ ID NO: 8 - DNA, 18 bp, artificial sequence
  primer
gttcgcctaatagtcaca

SEQ ID NO: 9 - DNA, 20 bp, artificial sequence
  probe
ctcatggcgctgctccgacg

SEQ ID NO: 10 - DNA, 20 bp, artificial sequence
  primer
ttgcagtccataaacccaca

SEQ ID NO: 11 - DNA, 18 bp, artificial sequence
  primer
gaagccaagagcagagca

SEQ ID NO: 12 - DNA, 20 bp, artificial sequence
  primer
tcaacccaccaaaacaacaa

SEQ ID NO: 13 - DNA, 20 bp, artificial sequence
  primer
agggttctcccaatcttcgt

SEQ ID NO: 14 - DNA, 20 bp, artificial sequence
  primer
agctgcttcctgcttgtctc

SEQ ID NO: 15 - DNA, 20 bp, artificial sequence
  primer
ggtctttgacggcttttgag

SEQ ID NO: 16 - DNA, 20 bp, artificial sequence
  primer
cctactgcttctgtcaaacc

SEQ ID NO: 17 - DNA, 20 bp, artificial sequence
  primer
tgtgggtttatggactgcaa

SEQ ID NO: 18 - DNA, 20 bp, artificial sequence
  primer
agctgcttcctgcttgtctc

SEQ ID NO: 19 - DNA, 20 bp, artificial sequence
  primer
gcacaatgaagcacacatcc

SEQ ID NO: 20 - DNA, 20 bp, artificial sequence
  primer
ccagttcccaaatccgagaa

SEQ ID NO: 21 - DNA, 25 bp, artificial sequence
  primer
ctggtctgagaagaggtttcatgag

SEQ ID NO: 22 - DNA, 18 bp, artificial sequence
  primer
catcctcaccctgaagta

SEQ ID NO: 23 - DNA, 18 bp, artificial sequence
  primer
acacgcagctcattgtag

SEQ ID NO: 24 - DNA, 20 bp, artificial sequence
  probe
cccatcgagcacggcatcgt

SEQ ID NO: 25 - DNA, 20 bp, artificial sequence
  primer
acatccaaggaaggcagcag

SEQ ID NO: 26 - DNA, 19 bp, artificial sequence
  primer
ttcgtcactacctccccgg

SEQ ID NO: 27 - DNA, 22 bp, artificial sequence
  probe
cgcgcaaattacccactcccga

SEQ ID NO: 28 - DNA, 18 bp, artificial sequence
  primer
cgcatgcgtccatttata

SEQ ID NO: 29 - DNA, 18 bp, artificial sequence
  primer
gtaggtttcagttgtttg

SEQ ID NO: 30 - DNA, 20 bp, artificial sequence
  primer
gcaaggtctttgacggcttt

SEQ ID NO: 31 - DNA, 18 bp, artificial sequence
  primer
ggtcatcttctcgcggtt

SEQ ID NO: 32 - DNA, 19 bp, artificial sequence
  primer
ggactcattccaattacag

(19) United States
(12) Patent Application Publication
(10) Pub. No.: US 2012/0058477 A1
(43) Pub. Date: Mar. 8, 2012

(54) METHOD FOR DETECTING THE EXPRESSION OF CYCLIN B2 GENE BY REAL-TIME QUANTITATIVE PCR

(76) Inventor: Xiaoming Dong, Beijing (CN)

(21) Appl. No.: 12/741,656

(22) PCT Filed: Nov. 7, 2008
(86) PCT No.: PCT/CN08/72982

§ 371 Date: May 6, 2010

(30) Foreign Application Priority Data
Nov. 9, 2007 (CN) 200710166283.7

(51) Int. Cl. C12Q 1/68

(52) U.S. Cl. 435/6.12

(57) ABSTRACT

A method and a kit for quantitative detection of the expression of cyclin B2 gene in blood samples, especially in serum.

### CROSS REFERENCE TO RELATED APPLICATIONS

This national phase entry application claims priority to international application PCT/CN2008/072982 filed on Nov. 7, 2008, and Chinese patent application 200710166283.7 filed on Nov. 9, 2007.

### TECHNICAL FIELD

The invention relates to the medical oncology, in particular, relates to a new method for detecting the expression of cyclin B2 gene in blood, especially serum, as well as a kit for this method.

### BACKGROUND

Malignant tumor is an important disease threatening human health. Presently, nearly 7 million people die from malignant tumor each year, in which about 1.3 million in China. Malignant tumor has been the second leading cause of death, after the cardiocerrebral vascular disease. More than 90% malignant tumors belong to solid tumors derived from epidermis (commonly known as cancers, such as lung cancer, liver cancer and stomach cancer etc.) The major reason that these malignant solid tumors (hereafter referred as tumors) cause death is metastasis, especially systemic metastasis. Clinically, it can be observed that, after the radical surgery, some of the early tumor patient are finally dead from systemic metastasis, even they have no lymph nod metastasis in pathology detection, which suggest that a little cancer cells have been spread in the body when taking surgery, but they can not be detected by current diagnosis means (such as medical imaging and nuclear medical methods etc.). These occult metastasis (also known as micrometastasis) is an important cause of recurring metastasis of tumor after sugery⁽¹⁾.

The animal experiments reveal that, when the weight of tumor reaches 1 gram (1 cm in diameter), there are about 10⁶ cancer cells which deviate from primary tumor and enter into blood every day. These cancer cells are seldom dead in the blood. After these cancer cells are transferred into organs by blood circulation, they soon penetrate the vessel epidemic barrier and enter into tissues, wherein 98% of the cancer cells apoptosis in tissues and are eliminated by the body, but there are still 2% of cancer cells survive and become metastasis focus⁽²⁾. Some German researchers have cultivated micromatastasis cancer cells, and proved that these cancer cells can grow in vitro, and the cancer cells which grow faster have shorter living period. Therefore, the presence of cancer cells in blood is an early indication of tumor metastasis, which indicates that the risk of metastasis is high. Establishing an early warning system for tumor metastasis will have great clinical value, because such system can help to prognose, and more importantly, it can achieve early diagnosis and early treatment for the metastasis after surgery, so as to maintain the effect of treatment and improve prognosis. Moreover, detecting the cancer cells in blood can serve as a molecular indication for chemotherapy of late stage tumor. It is reported that the patient with no cancer cells in the blood after chemotherapy has a favorable prognosis, and longer survive period^{(2,3)}.

Almost all tumors have an essential common feature, i.e. destroying the mechanism of cell cycle regulation will cause uncontrolled growth of the cells. Cyclins is the functional protein regulating cell cycle. They can be roughly divided into two major classes: G1 cyclins (or START cyclins), and mitotic cyclins. In mammals, G1 cyclins include cyclin D and cyclin E, and mitotic cyclins include cyclin A, B1 and B2, which are very stable during the whole interphase, but they are rapidly and specifically degraded when mitosing. Cyclin A and cyclin B have an important synergy effect for initiating mitosis. Once entered into mitosis stage, cyclin B will become the most important one. It will degrade at the interim between the metaphase and anaphase of the mitosis, and it is essential for completing mitosis. Cyclin B2 is synthesized depending on cell cycle. It links with cyclin-dependent kinase 1(cdc2) to form a complex which has positive regulation effect to cell mitosis, and it is critical to mitosis^{(4,5)}. As₂O₃ is a carcinogen which can promote cell propagation and induce tumorgenesis, but there exists uncertainty extrapolated from dose effect. Cyclin B2 has maximum expression at G2 phase, and is regulated at transcriptional level⁽⁶⁾. The expression level of it is high in the tumors such as lung cancer, breast cancer etc. It can be deemed as a good marker for serum diagnosis.

Therefore, in the past ten years, researchers worldwide have paid much attention to the detection of tumor markers in the blood⁽⁸⁾. Because of the development of modern immunology and molecular biotechnology, the mRNA of trace tumor marker in the blood can be detected now. Table 1 shows the results of the detection of tumor markers in the blood of patients with 6 types of common tumors. There are 6 reports annexed with follow-up results over two years, proving that the presence of cancer cells in the blood is negatively correlated with prognosis. According to the recent statistic results of the Beijing Oncology Institute, these tumors are accounted for the top ten malignant tumors in Beijing, and cover more than 70% of the total malignant tumor morbidity.

TABLE 1
The detection results of the tumor markers in the blood of malignant solid
tumor patient

| | | | | num- | | prog- |
|---|---|---|---|---|---|---|
| | | | | ber | posi- | nostic |
| | type of | tumor | method of | of | tive | signi- |
| the first writer | tumor | marker | detection | cases | rate | ficance |
| Yamashita^{(8,10)} | lung | CEA | RT-PCR | 103 | 62.1 | 0.0001 |
| | cancer | | | | | |
| Dong | lung | CK, S5A | FCM | 31 | 48.4 | 0.023 |
| Qianggang⁽⁹⁾ | cancer | | | | | |
| Yeh⁽¹⁰⁾ | stomach | CK19 | RT-PCR | 34 | 20.6 | 0.014 |
| | cancer | | | | | |
| Weigelt⁽¹¹⁾ | breast | CK19 etc. | RT-PCR | 94 | 31.0 | 0.0053 |
| | cancer | | | | | |
| Guller⁽¹²⁾ | colon | CEA, | RT-PCR | 39 | 28.2 | 0.035 |
| | cancer | CK20 | | | | |
| Ito⁽¹³⁾ | colon | CEA | RT-PCR | 99 | 18.7 | 0.03 |
| | cancer | | | | | |
| Mou⁽¹⁴⁾ | liver | MAGE | RT-PCR | 30 | 63.3 | — |
| | cancer | | | | | |
| Judson⁽¹⁵⁾ | ovary | EPCAM | IHC | 64 | 18.7 | — |
| | cancer | | | | | |

Note:
CEA: carcino-embryonic antigen;
CK: cytokeratin;
S5A: an antigen which can be recognized by lung cancer specific monoclonal antibody;
MAGE: melanoma-associated antigen;
EPCAM: epithelial cell adhesion molecule;
RT-PCR: reverse transcription polymerase chain reaction;
FCM: flow cytometry;
IHC: immunohistochemistry.

The current technology for detecting tumor markers in the blood is still not perfect and it has many problems. First, the methods of detection are not unified, including that the detection technologies and tumor markers used by the manufactures are different, so that it is difficult to compare the results. Second, the sensitivity is low, so that some of the patients may be missed diagnosed. Third, most of tumor marker genes have “Illegitimate Exprssion” phenomena in normal leucocyte, which may lead pseupositive result in peripheral blood in some healthy humans when detecting CK-19 mRNA using RT-PCR⁽¹⁰⁾.

Therefore, there exists a great need in the art for the method of detection with high sensitivity, good specification and wide applicability.

### SUMMARY

To achieve the above object, in the first aspect, the present invention provides an in vitro method for detecting cyclin B2 in a sample, comprising:
preparing a standard based on nucleotides 1078-1316 segment of cyclin B2 gene;
designing primers and Taqman probes based on nucleotides 1144-1243 segment of cyclin B2 gene;
relatively quantitative analyzing the relative expression amount of the mRNA of cyclin B2 gene using Taqman technology.

In the second aspect, the present invention provides a method for real-time quantitative determining the expression of cyclin B2 gene in a blood sample, comprising:
separating serum from the blood sample using separating gel;
extracting RNA from serum and obtaining cDNA;
preparing a standard based on nucleotides 1078-1316 segment in cyclin B2 gene;
designing primers and Taqman probes based on nucleotides 1144-1243 segment in cyclin B2 gene; and
relatively quantitative analyzing the relative expression amount of the mRNA of cyclin B2 gene using Taqman technology.

In the third aspect, the present invention provides a kit for quantitatively detecting the expression of cyclin B2 gene in a serum sample, comprising primers, DNA standard and Taqman probes, wherein said primers and Taqman probes are designed based on nucleotides 1144-1243 segment in cyclin B2 gene, and said standard is prepared based on nucleotides 1078-1316 segment in cyclin B2 gene.

After separating serum from blood using separating gel, the method of the present invention detects the expression of mRNA of cyclin B2 in the serum by real-time quantitative PCR, thereby determine the content of cyclin B2 in the serum.

The advantageous of the present invention are: first, it is suitable for the detection of all malignant tumors, and therefore can be used widely; second, it has higher specificity and sensitivity.

### BRIEF DESCRIPTION OF THE DRAWINGS

FIG. 1 shows the mean value of the relative expression amount of 10 various cancer detections listed in Table 2.

FIG. 2 shows the comparison of the expression levels of serum cyclin B2 before and after the treatment of cancers.

FIGS. 3A and 3B show the fluorescence quantitative PCR test for cyclin B2 in suspension of human cell line 293T (FIG. 3A) and total RNA extracted from human cell line 293T (FIG. 3B).

### DETAILED DESCRIPTION

The present invention provides a set of new technical solutions to solve the above problems existing in the art.

First, the present invention chooses cyclin B2 as the serum molecular marker which distinguish between tumor patient and normal human.

In the present invention, it is founded that cyclin B2 is highly expressed in the serum of some of the patients with solid tumors such as lung cancer, stomach cancer, liver cancer, breast cancer, esophagus cancer, pancreas cancer, colon cancer, prostate cancer, cervical cancer, ovary cancer, bladder cancer and kidney cancer etc. However, it is not expressed in 340 normal human cases.

A fundamental feature of tumor cell is unlimited propagation. Such feature is related with abnormal expression of the mRNA of cyclin B2. The malignant propagation of the malignant tumors are promoted by initiating the expression of cyclin B2 gene. Most of the adult human cells (except primary stem cell), germ cells and activated lymph cells, do not express cyclin B2, but the mRNA of cyclin B2 can be detected in some tumors. This makes cyclin B2 an ideal marker for tumor cells. Therefore, cyclin B2 can be used to identify normal cells and cancer cells. The phenotype of normal cells is cyclin B2 “−”, whereas that of cancer cells is cyclin B2 “+”. Secondly, it is also a difficult problem to identify normal leukocyte and cancer cell in the blood. Since the cancer cells are rare in the blood, it is almost impossible to find cancer cells only by pathomorphology observation. Presently, the analysis is usually conduced using high-sensitivity molecular detection technology such as RT-PCR. However, because normal leucocyte may illegally express tumor marker gene, it is difficult to obviate the pseudo-positive phenomenon. Separating serum can obviate the interference of the normal leucocytes during detection, eliminate psedo-positive result and improve diagnostic accuracy.

Real-time Quantitative PCR is a new method for detecting gene based on Taqman technology. The principle is hybridizing a set of primers which are specific to target gene with the fluorescent label and template cDNA, then hydrolyzing the fluorescent quench group at the 3′ end by the 3′ exonuclease activity of the Taq enzyme during polymerase chain reaction, so as to obtain fluorescence excitation signal, which is positively-related with the amount of template. Using this technology the mRNA of target gene can be detected from 1-10 pg RNA. Since a single cell contains at least 10 pg RNA, the detecting limit of real-time quantitative PCR can be 1-10 cells.

The present invention provides a relative quantitative real-time PCR technology to detect the expression of cyclin B2 gene. It mainly adopts the widely used 2^{-ΔΔCt} method to conduct the relative quantitative analysis⁽¹⁶⁾. The quantitative analysis is achieved by analyzing the ratio of target gene and internal-control gene (β-actin gene), and the ratio of target gene and internal-control gene (β-actin gene) in the standard, so as to obtain the expression level of the target gene relative to the internal-control gene, compared with the standard.

The present invention also provides several sets of PCR primers and probes, which can specifically amplify the mRNA of cyclin B2 and internal-control gene (β-actin gene). In particular, the inventor designed the primers and probes separately based on nucleotides 1144-1243 segment and nucleotides 1078-1316 segment of the cyclin B2 gene, using the primer design software available on the internet (such as Primer Express 2.0, ABI Inc.), based on the sequence of cyclin B2 gene (NM_{—}004701.2) and the internal-control β-actin gene (NM_{—}001101.2) which are disclosed in the Genbank database.

The present invention also provides a method of quantitatively detecting the relative expression amount of cyclin B2 gene in a biological sample using a standard. The standard of the present invention includes but not limited to a RNA sample extracted from human serum, whole blood or human cell line.

However, a skilled person in art will understand that the primers and probes listed herein are only exemplary. After reading this specification, he/she can synthesize the DNA standard within the nucleotides 1078-1316 segment of cyclin B2 gene by suitably choosing other primers. He/she can also choose other suitable primers and probes within nucleotides 1144-1243 of cyclin B2 gene, and use the relative quantitative 2^{-ΔΔCt} method, to achieve the purpose of the present invention.

Real-time quantitative PCR can be divided into two classes: relative quantification and absolute quantification. Relative quantification uses the cells in which target gene is positively expressed as the standard, and obtains the relative value of the expression of target gene in the tested sample by analyzing the ratio of target gene and internal-control gene (such as (β-actin gene). The advantageous of this method is that it can be easily developed, but the obtained value may be affected by positive standard cell. If different laboratories use different positive cells as the standard, the results are difficult to be compared.

Single Cell Quantitative Real-Time PCR

Comparing this method with traditional PCR, it is proved that the sensitivity of Single Cell Quantitative Real-time PCR increases 100 folds. Moreover, the inventor analyzed the content of cyclin B2 mRNA in 10 normal lung tissues and 55 lung cancer tissues using relative quantitative method, so as to determine the standard expression value of cyclin B2 gene in normal lung tissues. According to this standard, the positive expression rate of cyclin B2 in lung cancer tissues is 69.1%.

TABLE 2
the relative expression amount of cycin B2 in the
serum of various tumor patients

| | | expression | |
|---|---|---|---|
| | | amount relative | |
| | | to β-actin, | |
| | | compared | |
| | | with normal | |
| | | human samples | expression amount |
| | | (2^{-(Δ Δ Ct)} × | relative to β-actin |
| sample code | type of tumor | 100)* | (2^{-(Δ Ct)} × 100) |
| 070302XC03 | bladder cancer | 76573.4 | 0.06 |
| 070627XC16 | bladder cancer | 55264.1 | 0.13 |
| 070704XC01 | bladder cancer | 22.2 | 0.00 |
| 070127XC09 | bladder cancer | 21370.8 | 0.10 |
| | bladder cancer, after | | |
| | sugery of bladder | | |
| | cancer | | |
| 070109XC05 | colon cancer, after | 222409.1 | 1.73 |
| | sugery of colon | | |
| | cancer | | |
| 070522XC04 | lung cancer, | 34754.4 | 0.08 |
| | squamous cell | | |
| | carcinoma | | |
| 070620XC02 | bone metastasis of | 7.3 | 0.00 |
| | lung cancer | | |
| 070411XC09 | after surgery of | 540732.7 | 0.84 |
| | cervical cancer | | |
| 070627XC02 | stomach cancer | 0 | 0.00 |
| 070627XC07 | stomach cancer | 67013.1 | 0.47 |
| 070627XC13 | stomach cancer | 0 | 0.00 |
| 070522XC13 | stomach cancer | 13078.9 | 0.03 |
| 070612XC02 | After surgery of | 4598.4 | 0.01 |
| | hypopharyngeal | | |
| | cancer | | |
| 070426XC01 | lung cancer | 0 | 0.00 |
| 070601XC01 | lung cancer | 173461.5 | 1.35 |
| 070602XC02 | lung cancer | 523872.5 | 4.07 |
| 070127XC08 | lung cancer | 339206.9 | 2.64 |
| 070315XC02 | lung cancer | 0 | 0.00 |
| 070411XC10 | lung cancer | 1205713.9 | 9.38 |
| 070426XC02 | lung cancer | 774097.7 | 0.22 |
| 070508XC08 | lung cancer | 640374.4 | 0.18 |
| 070429XC01 | lung cancer | 115209.9 | 0.03 |
| 070123XC04 | lung cancer | 0 | 0.00 |
| 070127XC03 | lung cancer | 0 | 0.00 |
| 070308XC01 | lung cancer | 163956.8 | 0.12 |
| 070308XC03 | lung cancer | 27632.1 | 0.02 |
| 070508XC07 | lung cancer | 10340.4 | 0.01 |
| 070605XC02 | lung cancer | 0 | 0.00 |
| 070605XC06 | lung cancer | 0 | 0.00 |
| 070612XC01 | lung cancer | 98.9 | 0.00 |
| 070616XC09 | lung cancer | 25782.5 | 0.04 |
| 070616XC14 | lung cancer | 551693.0 | 0.78 |
| 070620XC01 | lung cancer | 67173.6 | 0.32 |
| 070620XC04 | lung cancer | 124782.5 | 0.60 |
| 070328XC06 | lung cancer | 1339.1 | 0.01 |
| 070529XC03 | lung cancer | 760.6 | 0.00 |
| 070626XC01 | lung cancer | 135300.4 | 0.21 |
| 070626XC02 | lung cancer | 141687.8 | 0.22 |
| 070626XC06 | lung cancer | 189043.2 | 0.30 |
| 070626XC09 | lung cancer | 0 | 0.00 |
| 070627XC03 | lung cancer | 24277.9 | 0.17 |
| 070627XC08 | lung cancer | 8837.7 | 0.06 |
| 070622XC01 | lung cancer | 196341.6 | 0.46 |
| 070627XC14 | lung cancer | 31957.3 | 0.08 |
| 070522XC05 | lung cancer | 20982.6 | 0.05 |
| 070710XC03 | lung cancer | 2355.6 | 0.10 |
| 070602XC01 | liver cancer | 0 | 0.00 |
| 070602XC03 | liver cancer | 0 | 0.00 |
| 070612XC04 | liver cancer | 0 | 0.00 |
| 070612XC10 | liver cancer | 523654.7 | 0.64 |
| 070616XC13 | liver cancer | 0 | 0.00 |
| 070328XC11 | liver cancer | 2769.9 | 0.00 |
| 070426XC05 | liver cancer | 4012.4 | 0.01 |
| 070612XC06 | thyroid cancer | 0 | 0.00 |
| 070710XC07 | after surgery of | 2036.4 | 0.09 |
| | thyroid cancer | | |
| 070429XC02 | after surgery of | 361843.5 | 0.10 |
| | thyroid cancer | | |
| 070710XC02 | after surgery of | 0 | 0.00 |
| | thyroid cancer | | |
| 070315XC01 | After surgery of | 36889.1 | 0.06 |
| | neck | | |
| | adenocarcinoma | | |
| 070428XC01 | lymphomas | 0 | 0.00 |
| 070627XC12 | lymphomas | 47711.7 | 0.34 |
| 070601XC02 | ovarian cancer | 678719.6 | 5.28 |
| 070328XC08 | ovarian cancer | 195255.8 | 1.52 |
| 070605XC05 | ovarian cancer | 0 | 0.00 |
| 070308XC02 | ovarian cancer | 0 | 0.00 |
| 070629XC05 | ovarian cancer | 0.3 | 0.00 |
| 070616XC10 | urinary tract | 862.5 | 0.00 |
| | infection | | |
| 070302XC05 | prostate cancer | 177678.1 | 0.13 |
| 070605XC04 | prostate cancer | 5215.4 | 0.00 |
| 070630XC02 | prostate cancer | 0 | 0.00 |
| 070426XC06 | esophageal cancer | 331076.8 | 2.58 |
| 070331XC02 | colon cancer | 518112.6 | 0.37 |
| 070612XC07 | colon cancer | 35044.6 | 0.04 |
| 070626XC08 | colon cancer | 101819.6 | 0.16 |
| 070411XC30 | breast cancer | 2160758.2 | 16.80 |
| 070429XC05 | breast cancer | 645902.2 | 0.18 |
| 070612XC05 | breast cancer | 27864.8 | 0.03 |
| 070620XC03 | breast cancer | 0 | 0.00 |
| 070323XC13 | breast cancer | 2489.7 | 0.01 |
| 070627XC06 | breast cancer | 43946.4 | 0.31 |
| 070627XC09 | breast cancer | 8829.7 | 0.06 |
| 070302XC13 | breast cancer | 250996.2 | 0.59 |
| 070522XC14 | breast cancer | 0 | 0.00 |
| 070629XC01 | breast cancer | 25.2 | 0.00 |
| 070704XC06 | breast cancer | 61.8 | 0.00 |
| 070710XC06 | breast cancer | 0 | 0.00 |
| 070109XC01 | breast cancer, after | 172550.1 | 0.09 |
| | sugery of breast | | |
| | cancer | | |
| 070116XC01 | after sugery of | 267773.9 | 2.08 |
| | breast cancer | | |
| 070116XC04 | after sugery of | 0 | 0.00 |
| | breast cancer | | |
| 070616XC12 | after sugery of | 7.1 | 0.00 |
| | breast cancer | | |
| 070123XC01 | after sugery of | 59866.3 | 0.09 |
| | breast cancer | | |
| 070323XC04 | after sugery of | 172.4 | 0.00 |
| | breast cancer | | |
| 070417XC02 | after sugery of | 41918.7 | 0.07 |
| | breast cancer | | |
| 070508XC02 | after sugery of right | 464801.8 | 0.25 |
| | breast cancer | | |
| 070612XC03 | colon cancer | 6485.9 | 0.01 |
| 070118XC05 | endometrial | 1342.4 | 0.01 |
| | carcinoma | | |
| 070627XC02 | stomach cancer | 0 | 0.00 |
| 070627XC07 | stomach cancer | 67013.1 | 0.47 |
| 070627XC13 | stomach cancer | 0 | 0.00 |
| 070522XC13 | stomach cancer | 13078.9 | 0.03 |
| 070711XC01 | pancreas cancer | 388352.2 | 16.31 |
| 070602XC05 | esophageal cancer | 0 | 0.00 |
| 070411XC18 | esophageal cancer | 835833.6 | 0.24 |
| 070508XC06 | esophageal cancer | 2217812.8 | 0.62 |
| 070508XC01 | esophageal cancer | 11573.2 | 0.01 |
| 070616XC15 | esophageal cancer | 402.4 | 0.00 |
| 070626XC07 | esophageal cancer | 210594.2 | 0.33 |
| 070627XC04 | esophageal cancer | 58326.2 | 0.41 |

*Relatively quantification, the baseline of normal human is 100.

The present invention will now be elucidated with reference to non-restrictive examples.

### Example 1

### Real-Time Quantitative PCR

Material and Method

Normal serum samples are obtained from the physical examination of new recruit in Military General Hospital of Beijing. Various tumor sera are mainly obtained from Chengde North Hospital and Baoding Hengxing Hospital. Trizol RNA extract kit is purchased from In Vitrogen Shanghai Shennengbocai Biotech. Inc. RevertAidT′ first chain cDNA synthesis kit is purchased from Promege Inc. PCR primers and probes are synthesized by Dalian Baoshengwu Biotech. Inc. Fluorescence quantitative PCR kit is purchased from Dalian Baosengwu Biotech. Inc. The mode of the quantitative PCR instrument is FB-2000 (Shanghai Fengling Biotech. Inc.).

Several sets of primers and probes are designed separately based on the sequence of cyclin B2 gene (NM_{—}004701.2) and 13-actin gene (NM_{—}001101.2) disclosed in the Genbank database, using the primer design software Primer Express 2.0 which is available on the internet, see Table 3.

TABLE 3
Primers and Taqman probes

| Target | | SEQ ID | |
|---|---|---|---|
| gene | group | No. | sequence |
| Cyclin B2 | Cyc B2-1 | SEQ ID | 5′CACAGGATACACAGAGAATG 3′ |
| gene | | NO: 1 | |
| | | SEQ ID | 5′CTTGATGGCGATGAATTTAG3′ |
| | | NO: 2 | |
| | | SEQ ID | 5′FAM-ATTGGAAGTCATGCAGCAC |
| | | NO: 3 | ATGGC-TAMRA3′ |
| | Cyc B2-2 | SEQ ID | 5′ GGACATTGATAACGAAGATTG |
| | | NO: 4 | 3′ |
| | | SEQ ID | 5′ GCTGCCTGAGATACTGAT 3′ |
| | | NO: 5 | |
| | | SEQ ID | 5′ |
| | | NO: 6 | FAM-AGAACCCTCAGCTCTGCAGT |
| | | | GAC -TAMRA 3′ |
| | Cyc B2-3 | SEQ ID | 5′ GGCACTCTTGCCTTC 3 |
| | | NO: 7 | |
| | | SEQ ID | 5′ GTTCGCCTAATAGTCACA 3′ |
| | | NO: 8 | |
| | | SEQ ID | 5′ |
| | | NO: 9 | FAM-CTCATGGCGCTGCTCCGACG- |
| | | | TAMRA 3′ |
| | Cyc B2-4 | SEQ ID | 5′TTG CAG TCC ATA AAC CCA |
| | | NO: 10 | CA3′ |
| | | SEQ ID | 5′GAA GCC AAG AGC AGA GCA |
| | | NO: 11 | GT3′ |
| | Cyc B2-5 | SEQ ID | 5′ TCA ACC CAC CAA AAC AAC AA |
| | | NO: 12 | 3′ |
| | | SEQ ID | 5′ AGG GTT CTC CCA ATC TTC GT |
| | | NO: 13 | 3′ |
| | Cyc B2-6 | SEQ ID | 5′ AGC TGC TTC CTG CTT GTC TC |
| | | NO: 14 | 3′ |
| | | SEQ ID | 5′ GGT CTT TGA CGG CTT TTG AG |
| | | NO: 15 | 3′ |
| | Cyc B2-7 | SEQ ID | 5′CCTACTGCTTCTGTCAAACC3′ |
| | | NO: 16 | |
| | | SEQ ID | 5′TGTGGGTTTATGGACTGCAA3′ |
| | | NO: 17 | |
| | Cyc B2-8 | SEQ ID | 5′ AGCTGCTTCCTGCTTGTCTC 3′ |
| | | NO: 18 | |
| | | SEQ ID | 5′ GCACAATGAAGCACACATCC 3′ |
| | | NO: 19 | |
| | Cyc B2-9 | SEQ ID | 5′CCAGTTCCCAAATCCGAGAA3′ |
| | | NO: 20 | |
| | | SEQ ID | 5′CTGGTCTGAGAAGAGGTTTCATG |
| | | NO: 21 | AG3′ |
| β-actin | β-actin-1 | SEQ ID | 5′ CATCCTCACCCTGAAGTA 3′ |
| gene | | NO: 22 | |
| | | SEQ ID | 5′ ACACGCAGCTCATTGTAG 3′ |
| | | NO: 23 | |
| | | SEQ ID | 5′ |
| | | NO: 24 | FAM-CCCATCGAGCACGGCATCGT- |
| | | | TAMRA 3′ |
| 18s rRNA | 18s rRNA- | SEQ ID | 5′ ACATCCAAGGAAGGCAGCAG3′ |
| | | NO: 25 | |
| | | SEQ ID | 5′ TTCGTCACTACCTCCCCGG3′ |
| | | NO: 26 | |
| | | SEQ ID | 5′ FAM- |
| | | NO: 27 | CGCGCAAATTACCCACTCCCGA-TA |
| | | | MRA 3′ |
| cyclin B2 | RT Cyclin | SEQ ID | 5′ CGCATGCGTCCATTTATA3′ |
| specific | B2-1 | NO: 28 | |
| reverse | RT Cyclin | SEQ ID | 5′ GTAGGTTTCAGTTGTTTG3′ |
| transcription | B2-2 | NO: 29 | |
| primers | RT Cyclin | SEQ ID | 5′ GCAAGGTCTTTGACGGCTTT3′ |
| | B2-3 | NO: 30 | |
| β-actin | RT β-actin-1 | SEQ ID | 5′ GGTCATCTTCTCGCGGTT 3′ |
| specific | | NO: 31 | |
| reverse | | | |
| transcription | | | |
| primer | | | |
| 18s rRNA | RT18s | SEQ ID | 5′ GGACTCATTCCAATTACAG 3′ |
| specific | rRNA-11 | NO: 32 | |
| reverse | | | |
| transcription | | | |
| primer | | | |

After extracting total RNA, read A260 and A280 using UV spectrophotometer (Nanodrop Inc., U.S.A.). Take 2 μg RNA, and synthesize cDNA according to the instruction of the kit. The reaction volume is 10 μl.

(1) Preparation of the Total RNA of the Test Sample

Collect blood using a tube with separating gel, centrifuge at 4,400 rpm for 10 minutes, move 250 μl blood into a 1.5 ml eppendorf tube, add 750 μl Trizol agent, vortex immediately, then shortly centrifuge, open the cover and add 200 μl chloroform, mix thoroughly, standard for 10 minutes, centrifuge at 12000 rpm for 10 minutes. Move the supernant (about 200 μl) into another 1.5 ml eppendorf tube, add equal value isopropanol, mix with the pellet for 1 hour, centrifuge at 12000 rpm for 10 minutes. Collect the pellet, wash with 1 ml 70% ethanol once, make the pellet dry, dissolve it in 10 μl DEPC-H₂O, take 3 μl to determine its concentration using UV spectrometer.

(2) Reverse transcription: dissolve 2 μg total RNA into 5.5 μl reaction volume, and conduct reverse transcription. Choose reverse transcription primer SEQ ID NO: 16 and SEQ ID NO: 19 which are specific to cyclin B2 and 13-actin. Reverse transcription are taken for each sample using MMLV.

(3) Quantitative PCR Reaction

Take reverse transcript product, dilute 10 times, using 5111 as template, conduct quantitative PCR, use 10×PCR buffer 2.5 μl.

TABLE 4
Calculation and arrangement of quantitative PCR reaction system

| | reagent | amount per tube (μl/tube) |
|---|---|---|
| calculation and | ddH₂O | 12.9 |
| arrangement | 10X buffer (Mg²⁺ free) | 2.5 |
| of the | Mg²⁺ (25 mM) | 3 |
| reaction | dNTP (10 mM) | 0.5 |
| system | F (25 mM) | 0.25 |
| | R (25 mM) | 0.25 |
| | Probe (25 mM) | 0.2 |
| | Taq enzyme | 0.4 |
| | aliquoted cDNA template | 5 |

| | |
|---|---|
| total | 25 μl/tube |
| protocol | 94° C. 3 min; 94° C. 15 sec, 59° C. 15 sec, |
| | 72° C. 12 sec, 40 cycles |

### Example 2

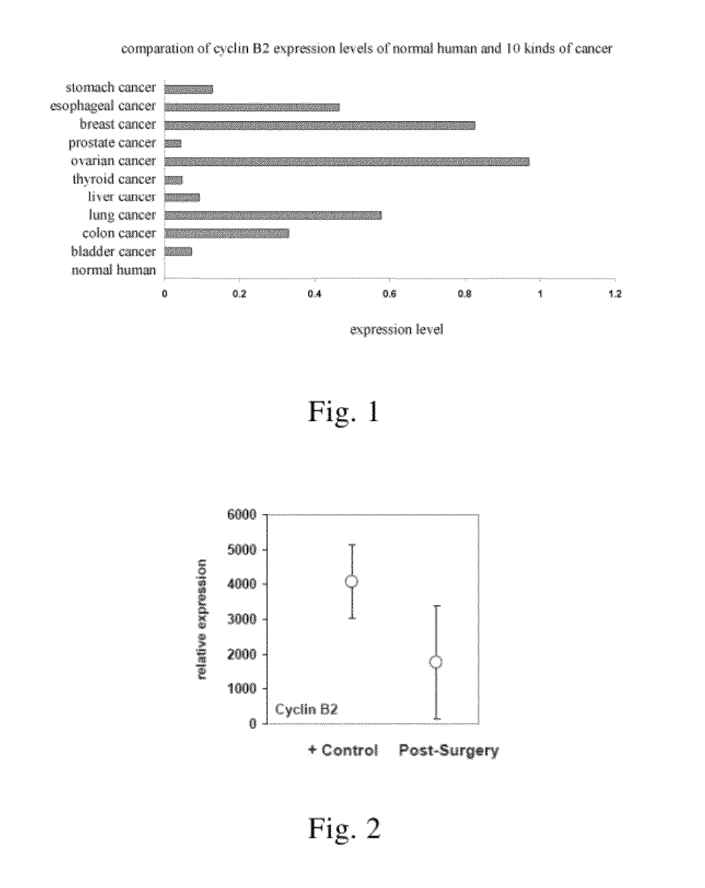

Use fluorescence quantitative PCR to test the use of serum 18S rRNA and cyclin B2 transcript in the cancer detection, determine Ct value of each tube, calculate the relative amount of cyclin B2 in the samples (FIG. 1 shows the mean value of the relative expression amount of 10 various cancer detections listed in Table 2).

Test various samples in Table 2 using the primers and probes listed in Table 3.

The error among different experiments is 5%. In all serum samples in 10 cancers (see Table 2) tested, it is found that cyclin B2 was overexpressed, compared with normal sample.

### Example 3

### Using Fluorescence Quantitative PCR Method to Test the Use of Serum 18S rRNA and Cyclin B2 Transcript in Treated Patient

To test whether the real-time PCR detection of serum expression level of cyclin B2 can be used to monitor the treatment of cancer (e.g. surgery, chemotherapy, radiotherapy etc.), and monitor the recrudesce of cancer after treatment, compare the expression levels of serum cyclin B2 before and after the treatment of cancers (FIG. 2). It is found that after the treatment (such as surgery), the expression levels of serum cyclin B2 in patients of aforesaid 10 types of cancers are lowered (the values showed in FIG. 2 are mean±SD).

### Example 4

### Using Fluorescence Quantitative PCR Method to Test 18S rRNA and Cyclin B2 Transcript in Human Cell Line, as Standard

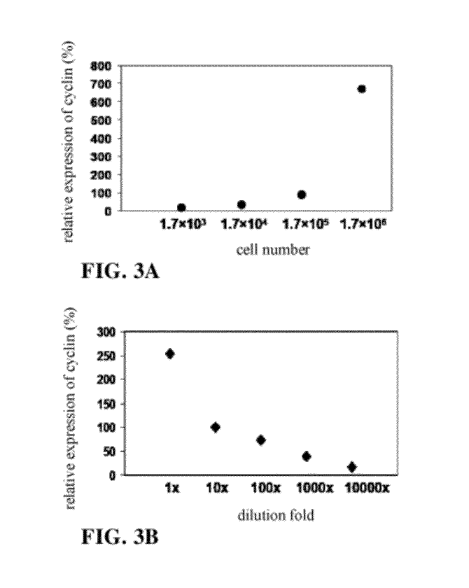

To choose a standard with high stability and high consistency to calculate the relative expression level of cyclin B2 in serum, so as to more accurately and more effectively reflect the effect of the treatment of cancer (such as surgery, chemotherapy, and radiotherapy etc.) and monitor the recrudesce of cancer after treatment, we gradiently diluted the suspension of human cell line 293T (FIG. 3A) and the total RNA extracted from human cell line 293T (FIG. 3B), and conduct quantitative PCR toward cyclin B2 using the method in Example 1 (FIG. 3 shows the mean values). It is founded that the relative expression level of cyclin B2 in 293T cell is in good correlation with cell number (FIG. 3A) and RNA concentration (FIG. 3B). Moreover, we repeated the above experiments many times in six month (once a week, all samples were kept under −80° C.), and found that the relative expression levels of cyclin B2 in all experiments are in good consistency and stability (SD<10%, data are not shown). These results shows that 18S rRNA and cyclin B2 transcript in human cell line tested by quantitative PCR method can serve as a standard.

While the invention has been disclosed in connection with certain embodiments and detailed descriptions, it will be clear to one skilled in the art that modifications or variations of such details can be made without deviating from the gist of this invention, and such modifications or variations are considered to be within the scope of the claims hereinbelow.

### REFERENCES

1. Zheng Yixing, Oncology, second edition, Beijng People's medical Publishing House, 2003, 151-152.
2. Hofmann H S, Hansen G, Burdach S, et al. Discrimination of human lung neoplasm from normal lung by two target genes. Am J Respir Crit Care Med. 2004; 170:516-519.
3. Li C, Shridhar K, Liu J. Molecular characterization of oncostatin M-induced growth arrest of MCF-7 cells expressing a temperature-sensitive mutant of p53 Breast Cancer Res Treat. 2003; 80:23-37.
4. Yoshitome S, Furuno N, Hashimoto E, et al. The C-terminal seven amino acids in the cytoplasmic retention signal region of cyclin B2 are required for normal bipolar spindle formation in Xenopus oocytes and embryos. Mol Cancer Res. 2003; 1:589-597.
5. Zhao S, Tsuchida T, Kawakami K, et al. Effect of As₂O₃ on cell cycle progression and cyclins D1 and B1 expression in two glioblastoma cell lines differing in p53 status. Int J Oncol. 2002; 21:49-55.
6. Wasner M, Haugwitz U, Reinhard W et al. Three CCAAT2 boxes and a single cell cycle genes homology region (CHR) are the major regulating sites for transcription from the human cyclin B2 promoter. Gene, 2003, 17(312):225.
7. Yamazaki K, Abe S, Takekawa H, et al. Tumor angiogenesis in human lung adenocarcinoma. Cancer, 1994, 74 (8):2245.
8. Kurusu Y, Yamashita J, Ogawa W. Detection of circulating tumor cells by reverse transcriptase-polymerase chain reaction in patients with respectable non-small cell lung cancer. Surgery, 1999, 126(5): 820-826.
9. Dong Qianggang, Sha Huifang, Quantitative analysis of lung cancer cells in peripheral blood cycle using flow cytometry, Tumor, Jan. 20, 2000 P 31-34.
10. Yeh K H, Chen Y C, Yeh S H, et al. Detection of circulating cancer cells by nested reverse-transcription polymerase chain reaction of cytokeratin 19(K19)-possible clinical significance in advanced gastric cancer[J]. Anticancer Res, 1998, 18(2B):1283-1286.
11. Weigelt B, Bosma A J, Hart A A M, et al. Marker genes for circulating tumour cells predict survival in metastasized breast cancer patients[J]. Br J Cancer, 2003, 88 (7): 1091-1094.
12. Guller U, Zajac P, Schnider A, et al. Disseminated single tumor cells as detected by real time quantitative polymerase chain reaction represent a prognostic factor in patients undergoing surgery for colorectal cancer[J]. Ann Surg, 2002, 236(6):768-776.
13. Ito S, Nakanishi H, Kodera Y, et al. Prospective validation of quantitative CEA mRNA detection in peritoneal washes in gastric carcinoma patients[J]. Br J Cancer, 2005, 93(9):986-992.
14. Mou D C, Cai S L, Peng J R, et al. Evaluation of MAGE-1 and MAGE-3 as tumour-specific markers to detect blood dissemination of hepatocellular carcinoma cells. Br J Cancer 2002; 86(1):110-6.
15. Judson P. L. 1; Geller M. A.; Bliss R. L.; Boente M. P.; Downs L. S.; Argenta P. A.; Carson L. F Preoperative detection of peripherally circulating cancer cells and its prognostic significance in ovarian cancer Gynecologic Oncology, Volume 91, Number 2, November 2003, pp. 389-394(6).

What is claimed is:

1. An in vitro method of detecting the expression of cyclin B2 gene in a sample, comprising:
preparing a DNA standard based on nucleotides 1078-1316 segment of cyclin B2 gene;
designing primers and Taqman probes based on nucleotides 1144-1243 segment of cyclin B2 gene; and
using Taqman technology to relatively quantitative analyse the mRNA of cyclin B2 gene in the sample.

2. A method according to claim 1, wherein said primers are selected from the group consisting of SEQ ID NO: 1, 2, 4, 5, 7, 8, 10-23, 25, 26 and 28-32.

3. A method according to claim 1, wherein said Taqman probes are selected from the group consisting of SEQ ID NO: 3, 6, 9, 24 and 27.

4. A method according to claim 1, characterized in that it further comprises:
separating serum from blood sample using separating gel; and
extracting RNA from serum and obtaining cDNA.

5. A kit for quantitatively detecting the expression of cyclin B2 gene in a serum sample, characterized in that it comprises primers, DNA standard and Taqman probes, wherein said primers and probes are designed based on nucleotides 1144-1243 segment of cyclin B2 gene, said DNA standard is prepared based on neucleotides 1078-1316 segment of cyclin B2 gene.

6. A kit according to claim 5, wherein said primers are selected from the group consisting of SEQ ID NO: 1, 2, 4, 5, 7, 8, 10-23, 25, 26 and 28-32.

7. A kit according claim 5, wherein said Taqman probes are selected from the group consisting of SEQ ID NO: 3, 6, 9, 24 and 27.

8. A kit according to claim 5, wherein said primer has the sequence of SEQ ID NO: 1 or 2.

9. A kit according to claim 5, wherein said probes has the sequence of SEQ ID NO: 3, and its 5′ end is linked with a fluorescence reporter group, and its 3′ end is linked with a fluorescence quench group.

10. A kit according to claim 5, wherein said DNA standard is obtained by amplification using probes which have the sequences of SEQ ID NO: 4 and 5.
